(19) United States
(12) Patent Application Publication
(10) Pub. No.: US 2024/0076742 A1
(43) Pub. Date: Mar. 7, 2024

(54) METHODS FOR IMPROVED CANCER TREATMENT

(71) Applicant: CELLPHENOMICS GMBH, Berlin (DE)

(72) Inventors: Christian Regenbrecht, Berlin (DE); Ulrike Pfohl, Berlin (DE); Jurgen Loskutov, Berlin (DE)

(73) Assignee: CELLPHENOMICS GMBH, Berlin (DE)

(21) Appl. No.: 17/795,312

(22) PCT Filed: Jan. 29, 2021
(86) PCT No.: PCT/EP2021/052148

§ 371 Date: Jul. 26, 2022

(30) Foreign Application Priority Data
Jan. 29, 2020 (DE) 10 2020 102 143.7

(51) Int. Cl. C12Q 1/6886; C12Q 1/6869

(52) U.S. Cl. CPC C12Q 1/6886; C12Q 1/6869; C12Q 2600/106; C12Q 2600/156

(57) ABSTRACT

A method for determination whether to start or to continue a treatment of cancer that includes: a) measuring at least one characteristic of at least one biomarker in at least one biological sample including tumor cells; b) determining a loss or mutation of at least one marker gene in the at least one biological sample by use of the at least one biomarker; c) determining to start or to continue treatment with the at least one inhibitor if the measurement indicates that the tumor cells in the at least one biological sample include the at least one marker gene whose mutational status indicates a favorable outcome, whereby the at least one inhibitor is selected specifically in view of the determination of the mutational status in the activity of transforming growth factor-β/bone morphogenetic protein (TGF-β/BMP) pathway. A biomarker corresponding to at least one marker gene and a use of the biomarker are also provided.

The present invention relates to improved methods for cancer treatment, specifically a method for determination whether to start or to continue a treatment of cancer with kinase inhibitors, especially the treatment by MEK inhibitors, which are widely used in oncology. Further, the present invention relates to a biomarker corresponding to at least one marker gene, especially SMAD4 gene, ARID1A gene, FBXW7 gene, BMPR2 gene, and a use of the biomarker in the inventive method.

Cancer is a term for a group of diseases in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue, such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord. For example, colorectal cancer (CRC) is the third most common cancer worldwide (WHO 2018). Colorectal cancer is generally used to refer to cancers of the large intestine, known as colon cancer, and rectum, known as rectal cancer. In summary, both cancer types are referred to as colorectal carcinoma. Tumors are classified into solid and non-solid tumors according to their localization and morphology. Solid tumors are an abnormal mass of tissue that usually does not contain cysts or liquid areas. Sarcomas are solid tumors and carcinomas also may be solid tumors. Therefore, solid tumors may be benign or malignant.

Cancer is a genetic disease and is caused by changes to genes that control the way cells function, especially how the cells grow and divide. Genetic changes that cause cancer can be inherited or acquired over a period of decades. It is known today that multiple genetic alterations including mutations in oncogenes, protooncogenes and tumor suppressor genes, such as p53, KRAS and KLF6, must accumulate in order to transform a normal cell into a malignant tumor cell. In particular, mutations in the genes controlling cell division, differentiation and tissue homeostasis lead to malignant tumor cells transformation. These malignant tumor cells exhibit increased cell growth, also known as proliferation, and the ability to invade surrounding tissue. These properties primarily define the malignant status of a tumor. The main cause of death in cancer is a metastatic disease. For instance, metastatic colorectal cancer (mCRC) is presented in 20% of patients and eventually develops in more than 30% of early-stage patients. Unfortunately, metastatic colorectal cancer is usually refractory to first line therapy agents and, despite the significant increase, for example to more than 30 months, of the median survival with the development of cytotoxic agents and introduction of the targeted therapy.

The biggest clinical challenge is the fact that tumor evolve, from benign over malignant to metastatic, as a heterogeneous entity, with a large intratumoral diversity. This intratumoral diversity is due to a broad variety of different mutations accumulating separately in the tumor cells. Usually, intratumoral diversity correlates with the tumor grade.

The publication Schumacher D, Andrieux G, Boehnke K, Keil M, Silvestri A, Silvestrov M, et al. (2019) “Heterogeneous pathway activation and drug response modelled in colorectal-tumor-derived 3D cultures” PLoS Genet 15(3), further referred to as Schumacher et al., discloses intratumoral heterogeneity as an underlying mechanism in differential drug response among parallel “sibling” 3D cultures established from a single colorectal tumor. Four of five sibling cultures showed resistance to epidermal growth factor receptor- (EGFR) inhibition and only one sibling culture was sensitive toward epidermal growth factor receptor- (EGFR), phosphoinositide 3-kinase (PI3K)-α and mTORC1/2 inhibition. When the exome and RNA was sequenced 646 differentially expressed genes were identified. Due to the heterogeneous mutational status of the sibling cultures from the same tumor, the drug response displayed substantial differences in IC₅₀ values of up to 1,000-fold for chemotherapeutic agents and targeted inhibitors.

Based on these findings, it is not surprising that already in the first treatment cycle with chemotherapy or targeted therapy a large number of tumors show resistance to first line therapy agents or develop such resistance during treatment.

The medicinal treatment for cancer can consist of chemotherapy and/or targeted therapy. In targeted therapy, processes inside the cell are specifically influenced. The two targeted therapies used in the treatment of colorectal cancer are angiogenesis inhibition and epidermal growth factor (EGF) receptor blocking, further referred to as EFGR. All these therapies do have significant side effects such as tiredness and exhaustion (fatigue), nausea up to and including vomiting, diarrhea, and inflammation of mucous membranes, blood count changes and fever. Due to the fact that the first line therapy remained unchanged over the last ten years, all colorectal cancer patients have to undergo these side effects even if the tumor is resistant to these first line therapy agents. The undesired side effects provoke an additional burden and stress for the patient suffering from a serve and often terminal disease, it would be very desirable to know in advance whether treating a specific patient with the chemotherapy and/or targeted therapy could really improve or even cure the patient's condition. Further on, the costs for the first line therapy have to be covered by the health care system whether the treatment is successful or not.

EP 2 443 252 B1 discloses a method to predict the likelihood that a patient suffering from KRAS wild type epidermal growth factor receptor (EGFR) expressing colorectal cancer will respond to the treatment with an anti-EGFR antibody comprising determining the expression level of prognostic genes or gene expression products thereof in a tissue sample as a biomarker.

For instance, a specific medication or a specific treatment, which may improve the clinical condition of a first patient, is less or not effective in a second patient suffering from the same type of cancer. Based on this, patients suffering from colorectal cancer may respond to a certain specific medication differently due to the different mutation pattern of their tumor cells. Although modern molecular biology and biochemistry has uncovered hundreds of genes whose activities influence the behavior of tumor cells, the state of their differentiation and their sensitivity or resistance to certain therapeutic medications, the status of these genes has generally not yet been exploited for the purpose of routine clinical decisions on medication treatments. Therefore, there is a need for diagnostic tests, methods and tools that use biomarkers which can simultaneously provide predictive information on the patient's response to different treatment options.

Thus, it would be advantageous to be able to assess the likelihood of success of the treatment with a diagnostic procedure before the first line treatment and to adjust the medication individually to each patient. This would not only increase the likelihood of successful treatment, but also reduce the number of unsuccessful treatments, thus saving the patient a higher quality of life and the health care system costs. Furthermore, a more concrete cohort stratification in research can be carried out and also leading to a more targeted development of new medicaments.

The object of the present invention is to provide a method for determination whether to start or to continue a treatment of cancer, particularly a solid tumor, such as colorectal cancer, which can be carried out by an analysis of the tumor with, preferred simple gene sequencing of nucleic acids isolated from the tumor, such as colorectal tumor cells by using an at least one biomarker to overcome the aforementioned disadvantages. The said at least one biomarker is also object of the present invention as well as a use of the at least one biomarker in the mentioned inventive method.

Said object of the present invention is solved by a method for determining whether to start or to continue a treatment of cancer comprising the provision of at least one inhibitor, the method comprising
a) measuring at least one characteristic of at least one biomarker in at least one biological sample comprising tumor cells of at least one tumor site of a tumor;
b) determining a loss or mutation of at least one marker gene in the at least one biological sample by use of the at least one biomarker;
c) determining to start or to continue treatment with the at least one inhibitor if the measuring indicates that the tumor cells in the at least one biological sample comprise the at least one marker gene whose mutational status indicates a favorable outcome, whereby the at least one inhibitor is selected specifically in view of the determination of the mutational status of the at least one marker gene;
   wherein the at least one marker gene is a tumor suppressor related to the activity of transforming growth factor-β/bone morphogenetic protein (TGF-β/BMP) pathway.

The mentioned cancer refers to a treatment of solid tumors. Solid tumors comprise sarcomas and carcinomas. Preferred is a treatment of head and neck, pancreas and/or colorectal cancer. Even more preferred is a treatment of colorectal cancer.

The mentioned treatment refers to a medication of cancer by targeted therapy and, in a further preferred embodiment, to the administration of said medication, examples of which have been set forth later herein. Targeted therapy is a type of treatment that uses drugs or other substances to identify and attack specific types of cancer cells with less harm to normal cells. Some targeted therapies block the action of certain enzymes, proteins, or other molecules involved in the growth and spread of cancer cells. Other types of targeted therapies support the immune system to kill cancer cells or deliver toxic substances directly to cancer cells. Targeted therapy may have fewer side effects than other types of cancer treatment. Most targeted therapies are either small molecule drugs or monoclonal antibodies. The treatment of the inventive method aims the transforming growth factor-β/bone morphogenetic protein (TGF-β/BMP) pathway further referred to as TGF-β/BMP pathway. TGF-β/BMP pathway influences the adhesion-dependent growth behavior of cells, which has an influence on wound healing and inflammation processes, cell differentiation and cell death, but also on cell motility and cell adhesion. The transmission of the signal via the TGF-β signaling pathway takes place between the plasma membrane and the nucleus of the cell by proteins of the SMAD family. However, there are numerous other signaling cascades that can also be SMAD independent, such as the phosphatidylinositol-3-kinase (PI3K)/AKT pathway. Mutations of these pathways may be harmless or even increase the ability of a tumor cell to grow without any natural regulation. Not only proliferation is regulated by this pathway, but also cell adhesion is influenced, in example the natural adhesion of the tumor cell is switched off and leads to the formation of metastases. The interaction of these signaling pathways and several mutations can lead not only to the formation of tumor cells but also, as disclosed in Schumacher et al., to complete or partial resistance to the targeted therapy.

An at least one biological sample comprises tumor cells, preferably colorectal cancer tumor cells, of at least one tumor site of a cancer tumor, preferably a colorectal cancer tumor. Preferably, the at least one biological sample comprises tumor cells of at least two tumor sites of a tumor. Thus, selecting at least two tumor sites of a tumor increases the possibility of obtaining a more complete sample of the intratumoral diversity of cancer mutations. A more complete sample of intratumoral diversity ensures that at least one biomarker provides a more validated indication of sensitivity to cancer treatment. Preferably, the biological sample comprises a mutation in the coding sequence of an at least one marker gene selected from the group comprising mothers against decapentaplegic homolog 4 (SMAD4) gene, AT-rich interactive domain-containing protein 1A (ARID1A) gene, F-Box/WD repeat-containing protein 7 (FBXW7) gene, bone morphogenic protein receptor type 2 (BMPR2) gene, Mitogen-activated protein kinase kinase (MEK also known as MAP2K) gene, and combinations thereof.

An at least one marker gene is a tumor suppressor. Preferably, at least one marker gene is selected from a group comprising mothers against decapentaplegic homolog 4 (SMAD4) gene, AT-rich interactive domain-containing protein 1A (ARID1A) gene, F-Box/WD repeat-containing protein 7 (FBXW7) gene, bone morphogenic protein receptor 2 (BMPR2) gene, Mitogen-activated protein kinase kinase (MEK) gene, and combinations thereof. The mothers against decapentaplegic homolog 4 gene, shortly described as SMAD4, is located in the chromosome 18q. The SMAD4 gene is among the top 10 most frequently mutated genes in colorectal cancer (CRC). The SMAD4 protein is an essential mediator in the TGF-β signaling pathway, which occupies a central position in the signaling networks that control cell growth and differentiation. The SMAD4 protein forms a heterocomplex with the receptor-regulated SMADs, translocate into the cell nucleus and is then recruited to the DNA by relevant SMAD-binding transcription factors.

The AT-rich interactive domain-containing protein 1A, shortly described as ARID1A gene, a homolog of yeast SWI1, encodes a large nuclear protein, p270, also known as BAF250a, which participates in forming a chromatin remodeling complex. The coordinated activity of the proteins of the SWI/SNF complex is responsible for the change in chromatin structure required to facilitate various cellular functions such as transcription, DNA synthesis and DNA damage repair. ARID1A is located at the chromosomal region 1p36.11. ARID1A has emerged as a candidate tumor suppressor based on its frequent mutations in cancer cells, such as ovarian clear cell and endometroid cancers. ARID1A participates in TGF-β/BMP pathway regulation through regulation of SMAD3 and SMAD5 expression. The F-Box/WD repeat-containing protein 7, shortly described as FBXW7 gene, also known as Fbw7, hCDC4, Ago and SEL10, is the substrate-recognition component of a specific ubiquitin ligase complex. The FBXW7 gene is located within a chromosomal region 4q31q, that is commonly deleted in cancers. Inactivating FBXW7 mutations have been found in many cancer cell-lines. FBXW7 is a tumor suppressor and targets cyclin E, c-Myc, Notch, c-Jun and sterol regulatory element-binding protein 1 (SREBP1) for degradation in a phosphorylation-dependent manner. The FBXW7 gene regulates TGF-β/BMP pathway by targeting for degradation corepressor TGF-β-induced factor 1 (TGIF1). TGIF1 recruits specific repressor complexes to SMAD2.

The bone morphogenic protein receptor type 2, shortly described as BMPR2 gene, is a transmembrane serine/threonine kinase receptor which is essential for embryogenesis, development, and adult tissue homeostasis. The BMPR2 gene locates within a chromosomal region 2q33.1-q33.2. BMPR2 is directly involved in BMP-branch signal transduction in TGF-β/BMP pathway. Upon BMP-induced heteromeric complex formation of BMPR2 with BMP type I receptor (BMPR1), BMPR2 activates BMPR1 by phosphorylation. The activated BMPR1 propagates the signal into the cell by phosphorylation of the SMAD1, SMAD5, and SMAD8 transcription factors. Expressions of some BMP proteins have been reported to be altered in CRC tissues.

Preferably, the at least one marker gene either has a mutation or has been lost through a mutation. The mutation as referred to herein is a mutation that causes loss of function of the encoded protein. Loss of function refers to a completely absent protein or a non-functional protein. The mutation can also cause significant reduction of protein activity, wherein the activity been reduced by more than 50% to 99% compared to the corresponding normal activity of the protein in a cell using any suitable protein activity assay well known in the art. Such reduction can be caused by a mutation in any of the binding pockets of the proteins involved in the pathways. The reduction in binding activity can be measured using routine methods known to one skilled in the art. A mutational status is the existence of a mutation in the at least one marker gene. The mutational status of a marker gene can be identified, preferred by sequencing a nucleic acid of a DNA, RNA, cDNA or a protein correlated with the at least one marker gene. The mutation may be present in the coding or non-coding region of the gene, and can be a deletion, insertion, splice-site mutation, a point mutation. There are several sequencing methods known in the art to sequence nucleic acids. One or more nucleic acid primers can be designed to bind any region of the target nucleic acid. For example, a nucleic acid primer can be designed to bind to a region comprising a potential mutation site or can be designed to complement the mutated sequence rather than the wild type sequence. Primer pairs can be designed to bracket a region comprising a potential mutation in a marker gene. A primer or primer pair can be used for sequencing one or both strands of DNA corresponding to the at least one marker gene. A primer can be used in conjunction with an at least one reagent, for example a nucleic acid probe or a hybridization probe, to amplify a region of interest prior to sequencing to boost sequence amounts for detection of a mutation in an at least one marker gene. Examples of regions which can be sequenced include an entire gene, transcripts of the gene and a fragment of the gene or the transcript, such as one or more of exons or untranslated regions. Examples of mutations to target for primer selection and sequence or composition analysis can be found in public databases which collect mutation information, such as COSMIC and dbGaP. Some mutations of the at least one marker gene, which can be associated with sensitivity to MEK inhibition, such as SMAD4, are listed in Table 2 in the Examples. More preferred is that the mutational status of the at least one marker gene is a mutation selected from the group consisting of deletion mutation, insertion mutation, frameshift mutant, nonsense mutant, missense mutant and splice mutant. Even more preferred is a loss-of-function mutation of the at least one marker gene. Especially, the inventors have discovered that the mutation SMAD4^{R361H} that was known to be associated with a loss of heterocomplex formation, which prevents it from accumulating in the cell nucleus, results in responsiveness to MEK inhibitors. A signaling network controls the activity of the TGF-β/SMAD pathway on several levels. The ERK MAP kinase phosphorylation attenuates the nuclear accumulation of the SMADs. The MAPK/ERK pathway is upregulated in SMAD4 mutant cells. Due to this, the at least one marker gene is related to the activity of TGF-β/BMP pathway. Further on, the heterogenic landscape of mutated SMAD4 gene within the same tumor, in this light, can assure very high degree of flexibility in tumor response to the chemotherapy and/or targeted therapy and give rise to a multi-drug resistant disease. Therefore, there is a critical need for recognizing therapeutic agents, capable of targeting specifically SMAD4 mutated tumors. It is known that SMAD4^{R361H} is associated with differential drug response towards epidermal growth factor receptor- (EGFR), MEK- and phosphoinositide 3-kinase (PI3K)-inhibitors. Especially, the functional loss of SMAD4 and thus loss of SMAD5 signaling renders SMAD4 mutation of tumor cells, especially colorectal tumor cells, more sensitive to MEK inhibitors. The inventors have discovered that the direct association of a loss of function mutation, such as the SMAD4^{R361H} mutation, leads to the sensitivity of a tumor carrying such mutation to, for example, MEK inhibitor. Thus, analysis of the functionality of this pathway in a tumor can be used in targeted therapy with kinase inhibitors, such as MEK inhibitors. For example, the SMAD4 gene or protein can be used as an at least one biomarker to assess an increased likelihood of success with a treatment with MEK inhibitor of cancer, such as head and neck, pancreas and/or colorectal cancer.

An at least one biomarker is a characteristic biological feature that can be used as a reference for processes and disease states in the body. Such an at least one biomarker can be genetic, anatomical and physiological or biochemical. Biomarkers must be objectively measurable quantities or structures. The at least one biomarker is the mutational status of the at least one marker gene, transcript or protein. Preferably, the at least one biomarker is a nucleic acid. In particular, the at least one biomarker is a nucleic acid. Even more preferred is that the at least one biomarker is selected from a group consisting of nucleic acid selected from the group consisting of DNA, mRNA and cDNA or any portion of any foregoing, wherein the portion corresponds to at least one mutation site of the at least one marker gene. The at least one biomarker is preferably corresponding to an at least one mutation site of the mothers against decapentaplegic homolog 4 (SMAD4) gene, AT-rich interactive domain-containing protein 1A (ARID1A) gene, F-Box/WD repeat-containing protein 7 (FBXW7) gene, and/or bone morphogenic protein receptor 2 (BMPR2) gene as the at least one biomarker. The mutation preferably leads to loss or loss-of-function of the gene product.

An at least one characteristic is the mutational status of the at least one marker gene or the functional and active marker gene itself and is used for the at least one biomarker. The at least one characteristic is selected to specifically identify the at least one marker gene and/or the mutation of the at least one marker gene. Therefore, the at least one characteristic is a nucleic acid or protein sequence of the at least one biomarker. Preferably, the at least one characteristic is selected from the group consisting of size, sequence, composition, and/or amount of the at least one biomarker. A possible at least one characteristic for the preferred genes is a mutation such as deletion, insertion, substitution. Even more preferred the at least one characteristic comprises SMAD4^{R361H}, identified through sequencing as SEQ-ID NO: 2 for the use of the at least one biomarker. The completely sequenced SMAD4 gene is shown in the sequencing protocol under SEQ ID NO: 2 as all the gene sequences mentioned below. A possible at least one characteristic for the preferred FBXW7 gene is a mutation such as deletion, insertion, substitution. Even more preferred the at least one characteristic comprises FBXW7^{R465H}, identified through sequencing as SEQ-ID NO: 6 for the use of the at least one biomarker. A possible at least one characteristic for the preferred ARID1A gene is a mutation such as deletion, insertion, substitution. Even more preferred the at least one characteristic comprises ARID1A^{Q51}*, identified through sequencing as SEQ-ID NO: 10 for the use of the at least one biomarker. A possible at least one characteristic for the preferred BMPR2 gene is a mutation such as deletion, insertion, substitution. Even more preferred the at least one characteristic comprises BMPR2^{R873}*, identified through sequencing as SEQ-ID NO: 14 for the use of the at least one biomarker.

Expression of the at least one biomarker is assessed by preparing mRNA and/or cDNA, in example a transcribed polynucleotide, from cells in an at least one biological sample, and by hybridizing the mRNA and/or cDNA with a reference polynucleotide as an isolated nucleic acid probe, for example a hybridization probe, which is a complement of an at least one biomarker nucleic acid, or a fragment thereof. The cDNA can, optionally, be amplified using any of a variety of polymerase chain reaction methods corresponding to the state of the art to hybridization with the reference polynucleotide. Expression of at least one biomarker likewise can be detected using quantitative PCR to assess the level of expression of the at least one biomarker. An example of the use of measuring mRNA levels is that an inactivating mutation in an at least one marker gene can result in an altered level of mRNA in a cell. The level can be upregulated due to feedback signaling protein production in view of nonfunctional or absent protein or downregulated due to instability of an altered mRNA sequence. Alternatively, any of the many known methods of detecting mutations or variants, for example single nucleotide polymorphisms, deletions, insertions and substitutions of an at least one biomarker may be used to detect occurrence of a mutation in an at least one marker gene in an at least one biological sample of a colorectal cancer tumor. The at least one biomarker is identified by an at least one reagent.

The at least one biomarker can be studied in combination with KRAS mutational status. Statistical methods can assist in the determination of treatment outcome upon measurement of the amount of the at least one marker by measurement of DNA or RNA. The amount of the at least one marker can be measured at multiple time points, for example before treatment, during treatment, after treatment with an at least one inhibitor, preferably a MEK inhibitor. A difference in amount from one time point to the next or from the at least one biological sample to the comparison sample without tumor cells can indicate prognosis of treatment outcome.

An at least one reagent refers to any molecule, which is capable of selectively binding to specifically indented target molecule, preferably the at least one biomarker. The at least one reagent can be derived from appropriate biological preparations. For purposes of detection of the target molecule, the at least one reagent may be specifically designed to be labeled by radioisotopes, chemiluminescent molecules or other state of the art labels. Molecules that can be utilized as at least one reagent are selected from the group consisting of RNA, DNA or nucleic acid sequences of RNA and DNA, primers, but are not limited to. Preferably, the at least one reagent is selected from the group consisting of nucleic acid sequences, primers and/or antibodies. Even more preferred is that the at least one reagent is at least one primer, which is complementary to the at least one marker gene and/or the at least one mutational site of the at least one marker gene. Complementary, in the broadest sense, refers to sequence complementarity between regions of two nucleic acid strands or between two regions of the same nucleic acid strand. One skilled in the art can readily design primers that selectively bind the target nucleic acid sequence. The at least one reagent is of sufficient length to selectively hybridize with the at least one marker gene or nucleic acid associated with the at least one marker gene, preferably the at least one reagent can bind to the nucleic acid of the at least one marker gene with base sequence specificity and remain bound, after washing. The at least one reagent can be used to aid in the isolation and sequencing of at least one marker gene nucleic acid sequence. The at least one reagent based on the sequence of a nucleic acid molecule of the at least one marker gene or the at least one mutational site of the at least one marker gene can be used to detect transcripts or genomic sequences corresponding to the at least one biomarker. Preferably, the at least one reagent comprises a label group attached thereto. Even more preferred the label group of the at least one reagent is selected of the group consisting of radioisotopes, fluorescent compounds, chemiluminescence molecules, enzymes and enzyme co-factors. Preferably, the at least one reagent comprises at minimum one or more nucleic acid primers designed to bind a region of the target nucleic acid in the at least one of SMAD4 gene, ARID1A gene, FBXW7 gene, BMPR2 gene. For example, the at least one reagent comprises, consists essentially of or consists of tools, such as primers, to characterize loss-of-function-causing mutations in at least one, two, three or all four of the genes selected from the group consisting of SMAD4 gene, ARID1A gene, FBXW7 gene, BMPR2 gene. Preferably, the at least one reagent comprises reagents for sequence analysis of at least SMAD4 gene. For example, a nucleic acid primer can be designed to bind to a region comprising a potential mutation site or can be designed to complement the mutated sequence rather than the wild type sequence. For example, primer pairs can be designed to bracket a region comprising a potential mutation in a marker gene. A primer or primer pair comprised in the kit can be used for sequencing one or both strands of DNA corresponding to the at least one marker gene. The at least one reagent may comprise a primer that can be used in conjunction with a compound, for example, a nucleic acid probe or a hybridization probe, to amplify a region of interest prior to sequencing to boost sequence amounts for detection of a mutation in an at least one marker gene. Further, the at least one reagent is even more preferred an antibody, which selectively binds to the at least one biomarker.

An at least one inhibitor is a retardant, specifically a substance that influences one or more reactions, chemical, biological or physical, in such a way that the reactions are slowed down, inhibited or prevented. Most of the inhibited reactions are enzyme reactions. In particular, the at least one inhibitor is preferably a signal transduction inhibitor that can interfere with or inhibit important cellular signal transduction pathways. Signal transduction in this context refers to the biochemical transfer of information from the cell membrane into the cell interior or from one cell compartment into another. The at least one inhibitor is used in the treatment of cancer, preferably colorectal cancer, in the targeted therapy and is the medicinal active compound to influence the proliferation of the tumor cells to lead to apoptosis of the tumor cells. Preferably, the at least one inhibitor is a MEK inhibitor. MEK inhibitors are antineoplastic agents that inhibit the function of the MAP kinase kinases MEK1 (MAP2K1) and MEK2 (MAP2K2). The classification of MEK inhibitors is that of protein kinase inhibitors. Since MEK inhibitors are bispecific protein kinases, they can be assigned to both tyrosine kinase inhibitors and serine/threonine kinase inhibitors. Further on, MEK inhibitors intervene in the signaling pathway by binding to MEK1 and MEK2. This prevents phosphorylation of transcription factors that depend on this step. This leads to the termination of transcription and inhibition of tumor cell proliferation. In particular, MEK inhibitors can be used for the therapy of malignant melanoma, but also for other tumors with BRAF mutations. MEK inhibitors are preferably selected from the group consisting of binimetinib, cobimetinib, selumetinib and trametinib. N-(3-{3-Cyclopropyl-5-[(2-fluor-4-iodphenyl)amino]-6,8-dimethyl-2,4,7-trioxo-3,4,6,7-tetrahydropyrido[4,3-d]pyrimidin-1(2H)-yl}phenyl)acetamid is the IUPAC name of trametinib. Even more preferred is that the MEK inhibitor is selected from a group comprising N-(3-{3-Cyclopropyl-5-[(2-fluor-4-iodphenyl)amino]-6,8-dimethyl-2,4,7-trioxo-3,4,6,7-tetrahydropyrido[4,3-d]pyrimidin-1(2H)-yl}phenyl)acetamid (Trametinib), [3,4-difluoro-2-[(2-fluoro-4-iodophenyl)amino]phenyl][3-hydroxy-3-(2S)-2-piperidinyl-1-azetidinyl]-methanone (cobimetinib), 5-[(4-Bromo-2-chlorophenyl)amino]-4-fluoro-N-(2-hydrokyethoxy)-1-methyl-1H-benzimidiazol-6-carboxamide (selumetinib), and mixtures thereof.

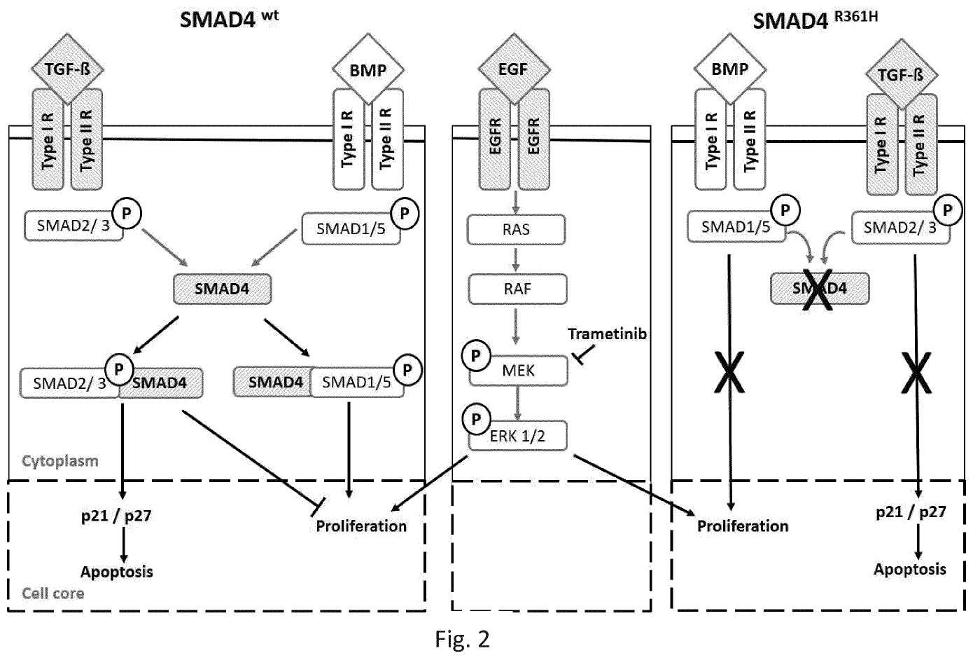

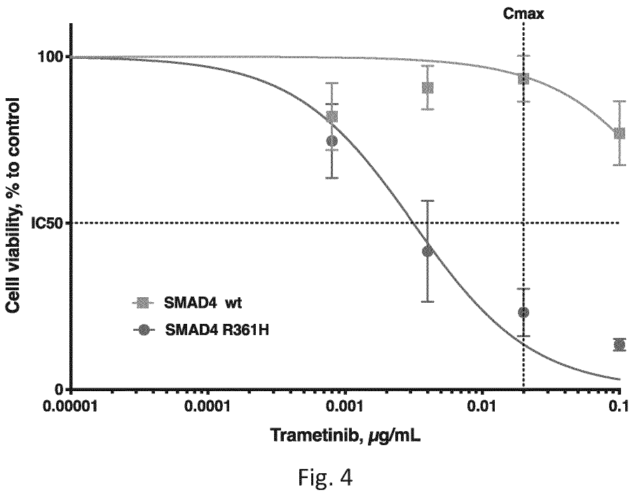

The inventor found that a loss-of-function mutation of SMAD4 gene increases the sensitivity to the MEK inhibitor therapy. As shown in the figures below, especially FIG. 4, the cell viability of colorectal cancer cells during a treatment with a MEK inhibitor, preferably trametinib, decreases if the SMAD4 gene is inactive as by a loss-of-function mutation such as SMAD4^{R361H} mutation SEQ-ID NO: 2. This is based on the interaction of the epidermal growth factor receptor- (EGFR) pathway and the transforming growth factor-β/bone morphogenetic protein (TGF-β/BMP) pathway. As shown in FIG. 2 below, both pathways, the EGFR pathway and the TGF-β/BMP pathway, control cell proliferation. If both pathways are active and the activity of the cell growth is increased by mutation a treatment with MEK inhibitors only interact with the EGFR pathway. Therefore, the uncontrolled increased proliferation may further take place through the TGF-β/BMP pathway. Based on the mechanism of action of SMAD4 in the signal transduction pathway, it is reasonable to extrapolate that loss of function of any protein that relates to the activity of TGF-β/BMP pathway, such as, ARID1A, FBXW7, BMPR2, MEK, and any combination thereof will result in similar sensitization to inhibitors, such as kinase inhibitors, preferably MEK inhibitors. Then the MEK inhibitor therapy may only lead to slower tumor growth but does not necessarily mean that the tumor stops growing completely or dies off. If the TGF-β/BMP pathway is disabled for example by a loss-of-function mutation, preferably by a SMAD4^{R361H} mutation SEQ-ID NO: 2, and a MEK inhibitor therapy targets the proliferation through the EGFR pathway the sensitivity to the MEK inhibitor is much higher. Further on, the intratumoral diversity leads to different mutational pattern by the same patient and in the same tumor site. Therefore, the same tumor may display tumor cells with an at least one mutation site in the at least one marker gene, preferably SMAD4 gene, and a fully functional at least one marker gene. Depending on the kind of mutation a MEK inhibitor therapy differentially targets the differently mutational tumor cells. On the one hand, tumor cells with an inactive SMAD4 gene are more sensitive to the therapy and will be erased, while on the other hand tumor cells with a fully active SMAD4 gene may slow down with the cell growth but will be resistant to the MEK inhibitor therapy. Due to this fact a tumor, such as a colorectal cancer tumor, which first responds well to the MEK inhibitor therapy may become resistant to the MEK inhibitor therapy. In this purpose the inventive method may give a reasonable tool to evaluate the likelihood of a positive outcome of the MEK inhibitor therapy. In addition, based on the result of the inventive method also new therapies may be implemented by targeting the interaction of the EGFR pathway and the TGF-β/BMP pathway. For the time being, however, this must remain the goal of further research.

Further provided is a method for treating cancer in a subject, the method comprising the steps of
(i) identifying a subject with at least one tumor that comprises at least one mutation site resulting in a disfunction of at least one tumor suppressor protein related to the activity of transforming growth factor-β/bone morphogenetic protein (TGF-β/BMP) pathway; and
(ii) administering to the subject at least one inhibitor.

Preferably, the subject is a mammal. Even more preferred a human.

The at least one tumor suppressor protein comprises at least one protein selected from the group comprising SMAD4, ARID1A, FBXW7, BMPR2, MEK, and any combination thereof. Preferably, the disfunction of the at least one tumor suppressor protein is selected from a group comprising a partial loss of function, complete loss of function, or the loss of the protein itself. A treatment, preferred kinase inhibitor treatment, even more preferred MEK inhibitor treatment responsive gene signature comprising loss of function of at least one marker gene or protein selected from SMAD4, ARID1A, FBXW7, BMPR2, MEK, and any combination thereof, is also provided.

Also provided is a method for treating cancer in a subject comprising at least one tumor site determined to comprise at least one mutation site, preferably a loss of function mutation, in at least one tumor suppressor protein related to the activity of transforming growth factor-β/bone morphogenetic protein (TGF-β/BMP) pathway, the method comprising administering at least one inhibitor to the subject identified as comprising at least one tumor site determined to comprise at least one mutation site, preferably a loss of function mutation, in at least one tumor suppressor protein related to the activity of transforming growth factor-β/bone morphogenetic protein (TGF-β/BMP) pathway.

An indication of treatment outcome can be assessed by studying the amount of the at least one biomarker either if the at least one marker gene is still active, lost or has a mutation. In the following, the three different possible alternative embodiments of the claimed method are disclosed in the following.

A first embodiment of the invention is defined by the at least one reagent, which is complement to the still active at least one marker gene of the at least one biomarker. On the one hand, the at least one reagent binds to the at least one characteristic of the at least one biomarker, the conclusion for the likelihood of the positive outcome of the treatment is less effective. On the other hand, the at least one reagent does not bind to the at least one marker gene of the at least one biomarker, the likelihood of a positive outcome of a treatment is higher due to the fact that the at least one marker gene does not have the sequence to provide normal activity.

A second embodiment of the invention is defined by the at least one reagent, which is complement to at least one mutation site of the at least one biomarker, whereby the conclusion for the likelihood of the positive outcome of the treatment is dependent on the mutation. If the mutation corresponds to a loss-of-function mutation, such as SMAD4^{R361H} mutation, the sensitivity to at least one MEK inhibitor increases and the likelihood of successful treatment of the colorectal cancer with the MEK inhibitor is very high.

A third embodiment of the invention is defined by at least two reagents are used for the inventive method. One of the at least two reagents is a complement to the still active at least one marker gene of the at least one biomarker. The other one of the at least two reagents is a complement to at least one mutation site of the at least one biomarker, preferably to a loss-of-function mutation, such as SMAD4^{R361H} mutation. The at least two reagents have different label groups selected from the group consisting of radioisotope, fluorescent compound, chemiluminescent molecules, enzyme, or enzyme co-factor. Due to the different label groups of the at least two reagents, it may be shown that:
a) only one of the at least two reagents bind to the at least one characteristic of the at least one biomarker; or
b) both of the at least two reagents bind to the at least one characteristic of the at least one biomarker; or
c) none of the at least two reagents bind to the at least one characteristic of the at least one biomarker.

In the case of a) the likelihood of the treatment of cancer is dependent of which of the at least two reagents bind to the at least one characteristic of the at least one biomarker. The conclusion is corresponding to the first and second application as described above. If b) occurs, no statement can be made about the likelihood of cancer being treated. However, therapy could be started and monitored to see how much the intratumoral diversity changes during therapy in order to be able to make a statement about the continuation of therapy. In the event of c) the at least one marker gene of the at least one biomarker is either lost or has a different at least one mutational site of the at least one biomarker. In this case the inventive method should be repeated with different at least one reagent to be able to give a verified decision about the start or continuation of the therapy.

The advantage of the inventive method is that a simple gene mutation causing loss of function of the tumor suppressor protein predicts favorable outcome to MEK inhibitor therapy before and during the application of the therapy. If the inventive method gives a negative result about the outcome of the MEK inhibitor therapy, the therapy may be adopted individually to the current patient without exposing the patient to a non-effective therapy and the undesired side effects. Furthermore, gene sequencing of the at least one biomarker can be applied within a few days and can already be carried out at low cost. Not only time, especially the patient's lifetime, is saved but also the costs of an ineffective MEK inhibitor therapy are saved to the health care system. Methods for monitoring tumor responsiveness to MEK inhibitors by sampling the tumor one or more times during the treatment for changes in the nucleic acids encoding the proteins are identified by the inventors as being markers for responsiveness or non-responsiveness to MEK inhibitor therapy. If one or more loss of function causing mutations are identified, then MEK inhibitor treatment can be started or continued. If such mutations are not identified, then MEK inhibitor therapy is likely unsuccessful, and the physician can select other treatments instead.

The present invention further relates to a biomarker to measure at least one characteristic of the at least one marker gene in a biological sample, wherein the at least one biomarker corresponds to at least one marker gene which is a tumor suppressor related to the activity of transforming growth factor-β/bone morphogenetic protein (TGF-β/BMP) pathway. The inventive biomarker is explicit described above.

The present invention further relates to a use of at least one in a method for determining whether to start or to continue a treatment of cancer with an at least one inhibitor as described above. Further on, the present invention also relates to a use of the at least one marker gene and the mutational status of the at least one marker gene as described above as an at least one biomarker.

The present invention further relates to a kit for testing tumor response to MEK inhibitor therapy comprising at least one reagent for detecting a loss-of-function mutation in nucleic acids isolated from at least one biological sample using at least one biomarker as described above which correlates to the at least one maker gene encoding a protein selected from the group comprising mothers against decapentaplegic homolog 4 (SMAD4) gene, AT-rich interactive domain-containing protein 1A (ARID1A) gene, F-box/WD repeat-containing protein 7 (FBXW7) gene, and/or bone morphogenic protein receptor 2 (BMPR2) gene, Mitogen-activated protein kinase kinase (MEK) gene, and combinations thereof.

The present invention further relates to a method of testing for a kinase inhibitor responsive solid tumor comprising detecting a loss of function in at least one marker gene encoding mothers against decapentaplegic homolog 4 (SMAD4), AT-rich interactive domain-containing protein 1A (ARID1A), F-box/WD repeat-containing protein 7 (FBXW7), bone morphogenic protein receptor 2 (BMPR2), Mitogen-activated protein kinase kinase (MEK), and combinations thereof, by contacting at least one biomarker isolated from at least one biological sample with a kit for testing for tumor response to inhibitor therapy as described above.

Methods and kits for identifying the kinase inhibitor responsive gene signature comprise, consists essentially of, or consists of reagents for sequence analysis of one, two, three or more of the nucleic acids encoding SMAD4, ARID1A, FBXW7, BMPR2, MEK, and any combination thereof. In one aspect, the gene signature comprises at least SMAD4. In one aspect, the loss of function is caused by SMAD4^{R361H} mutation.

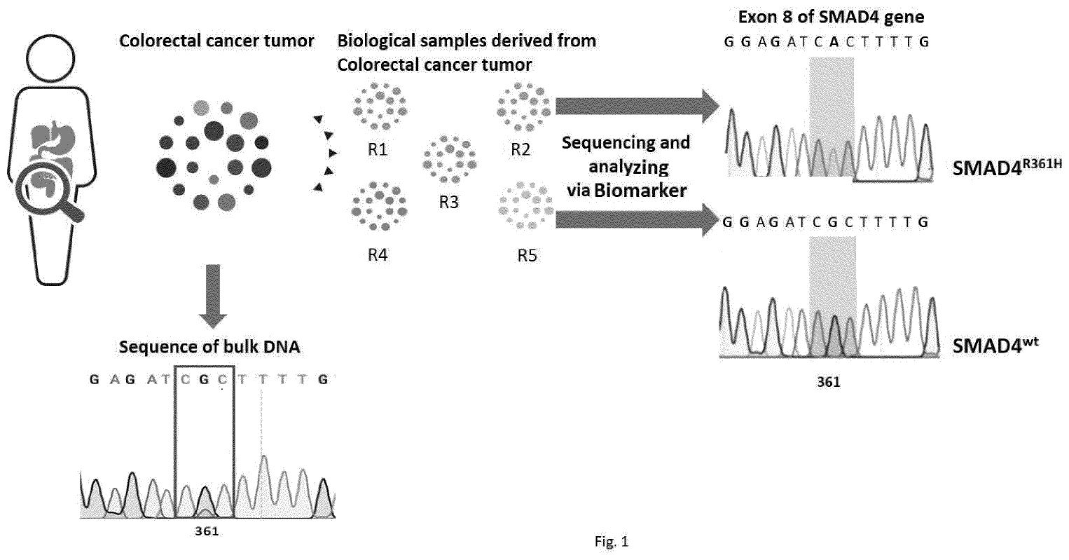

In the following, the invention will be descried in more detail with respect to the FIG. 1 to 4:

FIG. 1: shows a bulk sequencing of a tumor site might not display the intratumoral diversity, while at least two biological samples of the same at least one tumor site might show two different sequences of the same at least one marker gene, here SMAD4 gene.

FIG. 2: depicts different mechanism of action of the SMAD4^{wt} gene and SMAD4^{R361H} mutated protein in the TGF-β/BMP-pathway is shown in comparison with the EGFR-pathway. The wt in SMAD4^{wt} is used as “wild type” to indicate a non-mutated status of SMAD4 gene.

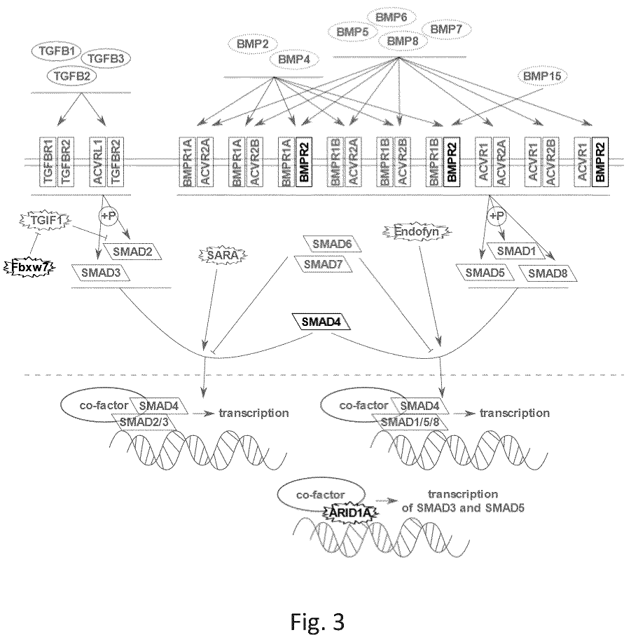

FIG. 3: is a schematic diagram of the TGF-β/BMP-pathway with the metabolic products of the SMAD4, ARID1A, FBXW7 and BMPR2 genes acting as marker genes, which are marked in bold.

FIG. 4: shows experimental results of the viability of colorectal cancer tumor cells with the SMAD4^{wt} or SMAD4^{R361H} mutation when treated with increasing concentration of trametinib used as a MEK inhibitor.

FIG. 1 shows schematically the intratumoral diversity of a tumor site by sequencing the SMAD4 gene. The collection of several biological samples, preferably single cell samples, increases the probability of detecting the intratumoral diversity of the tumor and being able to analyze it specifically. Bulk sequencing of the SMAD4 gene of a biological sample does not show the SMAD4^{R361H} mutation, whereas the single cell samples clearly show the SMAD4^{R361H} mutation. Therefore, a differentiated determination of the likelihood of a positive outcome of a targeted therapy, especially MEK inhibitor therapy, is only possible if intratumoral diversity is taken into account.

FIG. 2 shows the TGF-β/BMP-pathway and the EGFR-pathway, which both regulate the cell proliferation. Therefore, if both pathways work, a targeted therapy which influences only one of both pathways will not lead to apoptosis of the tumor cells. With knowledge which pathway is still active or if both are active, the targeted therapy can be adapted. Still the mutational status of tumor cells is patient individual, which requires quite precise diagnosis before individual adjustment to the targeted therapy can be applied.

FIG. 3 shows the detailed TGF-β/BMP-pathway, which regulates the cell proliferation and apoptosis. The proteins shown in FIG. 3 are the most important signalling proteins of the TGF-β/BMP-pathway. As already shown in FIG. 2, a loss-of-function mutation within the TGF-β/BMP-pathway can indicate whether treatment with MEK inhibitor is effective or not. Thus, all important proteins of the TGF-β/BMP-pathways can be indicators for a present dysfunction of the TGF-β/BMP-pathway. Thus, the proteins that can be considered as at least one marker gene are all those proteins that regulate the TGF-β/BMP-pathway significantly. Preferably SMAD4, ARID1A, FBXW7 and BMPR2 are used as the at least one marker gene. The SMAD4, ARID1A, FBXW7 and BMPR2 proteins are shown in bold in the diagram in FIG. 3. However, the at least one marker gene is not restricted to the preferred SMAD4, ARID1A, FBXW7 and BMPR2 proteins, but may also include other proteins of the TGF-β/BMP-pathway.

FIG. 4 shows that the tumor cell viability with the functional SMAD4^{wt} gene steadily lays over the mean inhibitory concentration (IC₅₀) by increasing concentrations of trametinib, which shows that the proliferation of the tumor cells with functional SMAD4 gene do not quite respond to a MEK inhibitor therapy. The mean inhibitory concentration, further referred to as IC₅₀, is the concentration of an inhibitor at which half-maximal inhibition is observed. The tumor cell viability of the loss-of-function SMAD4^{R361H} mutation SEQ ID NO: 2 already decreases below the IC₅₀ at a trametinib concentration of 0.006 μg/ml, which shows that the sensibility towards a MEK inhibitor therapy, especially trametinib, increases as the function of the SMAD4 marker gene is lost. For the experiment, the biological tumor samples were dissociated by incubation with TrypLE Express solution (ThermoFisher Scientific, Germany) and 1.5×10\hat{ }3 cells per well were seeded in four replicates in Matrigel and overlaid with culture medium. After 3-day incubation at 37° C. in 5% CO₂, medium was removed and fresh medium containing trametinib was added. Trametinib was tested in four 1:5 dilution concentrations. Following concentrations were tested: 0.1 μg/ml, 0.02 μg/ml, 0.004 μg/ml and 0.0008 μg/ml. Cells treated with DMSO were used as control while Matrigel only was used for background readout. Staurosporine was used as internal positive control. After 4-day treatment, cell viability was determined by CellTiterGlo® assay. Cell plating, treatment and testing were handled by Biomek FXP Liquid Handler (Beckman Coulter).

As far as the term weight percent or % by weight is used with respect to the components being comprised from the claimed composition, the term weight percent is referred to the amount of one or more components relative to the total amount of the composition throughout this specification, except where expressively stated otherwise. The expression “wt-%” is used throughout the present invention as an abbreviation for weight percent if not indicated otherwise.

In the context of the invention, the expressions “about” and “approximately” in connection with numerical values or ranges are to be understood as a tolerance range, which a person skilled in the art would consider as common or reasonable based on his or her general knowledge and in view of the invention as a whole. In particular, the expressions “about” and “approximately” refer to a tolerance range of ±20%, preferred ±10% and further preferred ±5% with respect to the designated value. The lower end values and the upper end values of the various ranges, especially the weight percent ranges, but not restricted thereto, claimed in the present invention may be combined with each other in order to define new ranges.

Further, in the context of the present invention, all references to standards, norms, or standardization protocols, for example ISO, ASTM etc., in connection with properties, numerical values or ranges referred to are to be understood as the latest updated version of said standard, norm, or standardization protocol being in force at the date of filling of the invention.

The present invention will be hereunder described in more detail with reference to the following non-limiting example in accordance with the present invention of the biomarker, the use of the biomarker in the inventive method to determinate, whether to start or to continue a treatment of colorectal cancer and the use of the at least one marker gene and the mutational status of the at least one marker gene as an at least one biomarker.

### EXAMPLE 1

Nucleic Acid Preparation

Genomic DNA from a biological sample BE1 and a comparative sample C1 were prepared using the QIAamp DNA Mini Kit (QIAGEN GmbH, QIAGEN Strasse 1, 40724 Hilden, Germany) according to the manufacturer's protocols. Total DNA was eluted in 30 μl DNase/RNase-free distilled water (Thermo Fisher Scientific, 168 Third Avenue, Waltham, MA USA 02451, United States of America). Isolated DNA was quantified using a NanoDrop© ND-1000 Spectrophotometer (NanoDrop Technologies, 3411 Silverside Rd, 19819 Wilmington, United States of America) and stored at −20° C. until further use.

The combinations of primers that can be used for diagnosis are the forward and reverse primers of gene SMAD4. Though it will be expected that since the genes SMAD4^{R361H}, shown in SEQ-ID NO: 2, lie in a contiguous sequence the forward primer SMAD4 fw2, shown in SEQ-ID NO: 3, and the reverse primer SMAD4 rev2, shown in SEQ-ID NO: 4, can amplify a product. The term fw or F is the abbreviation for forward and the term rev or R is the abbreviation for reverse.

TABLE 1
Primer sequences to identify a mutation of
SMDA4 gene

| | | Sequence |
|---|---|---|
| | | Identification |
| Name | Sequence 5′-3′ | Number |
| SMAD4 | AAATTCTCAGTTGACCTGGTCC | SEQ ID NO: 3 |
| fw2 (F) | | |
| SMAD4 | ACCGACAATTAAGATGGAGTGC | SEQ ID NO: 4 |
| rev2 (R) | | |

PCR

The PCR reaction mixture (25 μl) contained 100 ng isolated DNA, 1× Phusion GC buffer (ThermoFisher Scientific, Germany), 3% DMSO, 200 μM dNTPs, about 0.5 μM of each primer, and about 0.02 U/μl of Phusion DNA polymerase (ThermoFisher Scientific, Germany). Amplification starts with initial denaturation at 95° C. for 5 min. Amplification protocol contains of denaturation at 95° C. for 30 sec, primer annealing at 58° C. for 30 sec and extension at 72° C. for 30 sec. Steps within amplification protocol are repeated 34 times in an automated DNA thermal cycler (Biometra TRIO Thermal Cycler Series, Analytik Jena AG, Germany). Final extension is performed at 72° C. for 5 min. The PCR products are resolved on 2% agarose gel stained with Ethidium bromide. PCR products are visualized under the UV trans illuminator and are expected to be 688 bp. Molecular size markers (GeneRuler™ 100 bp DNA Ladder, ThermoFisher Scientific, Germany) are run concurrently. PCR products were sequenced using SMAD4 fw2 SEQ-ID NO: 3 and SMAD4 rev2 primer SEQ-ID NO: 4 (LGCgenomics, Berlin, Germany). Sequencing results were analyzed using CLC genomic workbench (Qiagen Bioinformatics, GmbH, QIAGEN Strasse 1, 40724 Hilden, Germany)).

According to the inventive method following steps were accomplished to determine whether to start a treatment of colorectal cancer comprising the provision of at least one inhibitor, preferably MEK inhibitor:

a) Information about the Biological Sample BE1 and the Comparative Sample C1

The biological sample BE1 and the comparative sample C1 were derived from the colon cancer of a Caucasian woman. At the time of surgery the patient was 47 years old. The chemo-naive tumor was graded and staged as pT3, pN0 (0/22), M1(HEP), L0, V0, R0.
Histopathology: G2 UICC IV
Molecular Pathology: KRAS mutated
Localization: colon sigmoideum (60 cm ab ano)
Tumor marker: EA 1.0 ng/ml, CA-19-9 13.5 U/ml
No family history of HNPCC

b) Sequencing the Tumor Cells and Using the SMAD4 Gen as an at Least One Biomarker

The sequencing of the BE1 and a comparative sample C1 were accomplished according to the before mentioned methods of nucleic acid preparation and PCR by using the primers P1 and P2 to identify if the biological sample BE1 and C1 have a mutation or not of the marker gene SMAD4.

TABLE 2
Identification of a mutation of the SMAD4
gene of biological example BE1

| | | | Sequence |
|---|---|---|---|
| | | | Identifi- |
| Sample | | | cation |
| Name | Name | Sequence 5′-3′ | Number |
| C1 | SMAD4 | | SEQ ID |
| | wt | | NO: 1 |
| BE1 | SMAD4 | | SEQ ID |
| | R361H | | NO: 2 |
| P1 | SMAD4 | AAATTCTCAGTTGACCTGGTCC | SEQ ID |
| | fw2 (F) | | NO: 3 |
| P2 | SMAD4 | ACCGACAATTAAGATGGAGTGC | SEQ ID |
| | rev2 (R) | | NO: 4 |

C1 is a comparison sample, means it is the SMAD4 wild type gene, which contains fully function and the sequence protocol SEQ-ID NO:1 showed no mutation of the marker gene SMAD4 at nucleic acid position 1082. The sequence protocol SEQ-ID NO:2 of BE1 showed a loss-of-function SMAD4^{R361H} mutation at nucleic acid position 1082.

c) Giving an Advice Whether to Start a Treatment

The sample BE1 showed a SMAD4^{R361H} mutation, which is a loss-of-function mutation of the SMAD4 gene. Therefore, a MEK inhibitor therapy, especially with trametinib, may be successful. The MEK inhibitor therapy should be initiated and the progress of the MEK inhibitor therapy should be monitored in regular time steps.

### EXAMPLE 2

Nucleic Acid Preparation

Genomic DNA from a biological sample BE2 and a comparative sample C2 were prepared using the QIAamp DNA Mini Kit (QIAGEN GmbH, QIAGEN Strasse 1, 40724 Hilden, Germany)) according to the manufacturer's protocols. The total DNA was eluted in 30 μl DNase/RNase-free distilled water (Thermo Fisher Scientific, Germany). Isolated DNA was quantified using a NanoDrop® ND-1000 Spectrophotometer (NanoDrop Technologies, 3411 Silverside Rd, 19819 Wilmington, United States of America) and stored at −20° C. until further use.

The combinations of primers that can be used for diagnosis are the forward and reverse primers of gene FBXW7. Though it will be expected that since the gene FBXW7, shown in SEQ-ID NO: 5, lie in a contiguous sequence the forward primer FBXW7 fw, shown in SEQ-ID NO: 7, and the reverse primer FBXW7 rev, shown in SEQ-ID NO: 8, can amplify a product.

PCR

The PCR reaction mixture (25 μl) contained 100 ng isolated DNA, 1× Phusion GC buffer (ThermoFisher Scientific, Germany), 3% DMSO, 200 μM dNTPs, about 0.5 μM of each primer, and about 0.02 U/μl of Phusion DNA polymerase (ThermoFisher Scientific, Germany). Amplification starts with initial denaturation at 95° C. for 5 min. Amplification protocol contains of denaturation at 95° C. for 30 sec, primer annealing at 56° C. for 30 sec and extension at 72° C. for 30 sec. Steps within amplification protocol are repeated 34 times in an automated DNA thermal cycler (Biometra TRIO Thermal Cycler Series, Analytik Jena AG, Germany). Final extension is performed at 72° C. for 5 min. The PCR products are resolved on 2% agarose gel stained with Ethidium bromide. PCR products are visualized under the UV trans illuminator and are expected to be 688 bp. Molecular size markers (GeneRuler™ 100 bp DNA Ladder, ThermoFisher Scientific, Germany) are run concurrently. PCR products were sequenced using FBXW7 fw SEQ-ID NO:7 and FBXW7 rev SEQ-ID NO:8 (LGC genomics, Berlin, Germany). Sequencing results were analyzed using CLC genomic workbench (Qiagen Bioinformatics GmbH, QIAGEN Strasse 1, 40724 Hilden, Germany)).

According to the inventive method following steps were accomplished to determine whether to start a treatment of colorectal cancer comprising the provision of at least one inhibitor, preferably MEK inhibitor:

a) Information about the Biological Sample BE2 and the Comparative Sample C2

The biological sample BE2 and the comparative sample C2 were derived from the colon cancer of a Caucasian woman. At the time of surgery the patient was 47 years old. The chemo-naive tumor was graded and staged as pT3, pN0 (0/22), M1(HEP), L0, V0, R0.
Histopathology: G2 UICC IV
Molecular Pathology: KRAS mutated
Localization: colon sigmoideum (60 cm ab ano)
Tumor marker: EA 1.0 ng/ml, CA-19-9 13.5 U/ml
No family history of HNPCC

b) Sequencing the Tumor Cells and Using the FBXW7 Gene as an at Least One Biomarker.

The sequencing of the BE2 and a comparative sample C2 were accomplished according to the before mentioned methods of nucleic acid preparation and PCR by using primers P3 and P4 to identify if the biological sample BE2 and C2 have a mutation or not of the marker gene FBXW7.

TABLE 3
Identification of a mutation of the FBXW7
gene of biological example BE2

| | | | Sequence |
|---|---|---|---|
| | | | Identifi- |
| Sample | | | caton |
| Name | Name | Sequence 5′-3′ | Number |
| C2 | FBXW7 | | SEQ ID |
| | wt | | NO: 5 |
| BE2 | FBXW7 | | SEQ ID |
| | R465H | | NO: 6 |
| P3 | FBXW7 | CAAACAGGAAGCTGACAACAC | SEQ ID |
| | fw (F) | | NO: 7 |
| P4 | FBXW7 | AGTGGGACAAATTCAGGCTTC | SEQ ID |
| | rev (R) | | NO: 8 |

C2 is a comparative sample, the FBXW7 wild type gene, which contains fully function and the sequence protocol SEQ-ID NO:5 showed no mutation of the marker gene FBXW7 at nucleic acid position 826. The sequence protocol SEQ-ID NO:6 of BE2 showed a loss-of-function FBXW7^{R465H} mutation at nucleic acid position 826.

c) Giving an Advice Whether to Start a Treatment

The sample BE2 showed a FBXW7^{R465H} mutation, which is a loss-of-function mutation of the FBXW7 gene. Therefore, a MEK inhibitor therapy, especially with trametinib, may be successful. The MEK inhibitor therapy should be initiated and the progress of the MEK inhibitor therapy should be monitored in regular time steps.

### EXAMPLE 3

Nucleic Acid Preparation

Genomic DNA from a biological sample BE3 and a comparative sample C3 were prepared using the QIAamp DNA Mini Kit (QIAGEN GmbH, QIAGEN Strasse 1, 40724 Hilden, Germany)) according to the manufacturer's protocols. The total DNA was eluted in 30 μl DNase/RNase-free distilled water (Thermo Fisher Scientific, Germany).

Isolated DNA was quantified using a NanoDrop© ND-1000 Spectrophotometer (NanoDrop Technologies, 3411 Silverside Rd, 19819 Wilmington, United States of America) and stored at −20° C. until further use.

The combinations of primers that can be used for diagnosis are the forward and reverse primers of gene ARID1A. Though it will be expected that since the gene ARID1A, shown in SEQ-ID NO: 9, lie in a contiguous sequence the forward primer AR-TID1A fw, shown in SEQ-ID NO: 11, and the reverse primer ARID1A rev, shown in SEQ-ID NO: 12, can amplify a product.

PCR

The PCR reaction mixture (25 μl) contained 100 ng isolated DNA, 1× Phusion GC buffer (ThermoFisher Scientific, Germany), 3% DMSO, 200 μM dNTPs, about 0.5 μM of each primer, and about 0.02 U/μl of Phusion DNA polymerase (ThermoFisher Scientific, Germany). Amplification starts with initial denaturation at 95° C. for 5 min. Amplification protocol contains of denaturation at 95° C. for 30 sec, primer annealing at 56° C. for 30 sec and extension at 72° C. for 30 sec. Steps within amplification protocol are repeated 34 times in an automated DNA thermal cycler (Biometra TRIO Thermal Cycler Series, Analytik Jena AG, Germany). Final extension is performed at 72° C. for 5 min. The PCR products are resolved on 2% agarose gel stained with Ethidium bromide. PCR products are visualized under the UV trans illuminator and are expected to be 688 bp. Molecular size markers (GeneRuler™ 100 bp DNA Ladder, ThermoFisher Scientific, Germany) are run concurrently. PCR products were sequenced using ARID1A fw SEQ-ID NO:11 and ARID1A rev SEQ-ID NO:12 (LGC genomics, Berlin, Germany). Sequencing results were analyzed using CLC genomic workbench (Qiagen Bioinformatics GmbH, QIAGEN Strasse 1, 40724 Hilden, Germany)).

According to the inventive method following steps were accomplished to determine whether to start a treatment of colorectal cancer comprising the provision of at least one inhibitor, preferably MEK inhibitor:

a) Information about the Biological Sample BE3 and the Comparative Sample C3

The biological sample BE3 and the comparative sample C3 were derived from the colon cancer of a Caucasian woman. At the time of surgery the patient was 47 years old. The chemo-naive tumor was graded and staged as pT3, pN0 (0/22), M1(HEP), L0, V0, R0.
Histopathology: G2 UICC IV
Molecular Pathology: KRAS mutated
Localization: colon sigmoideum (60 cm ab ano)
Tumor marker: EA 1.0 ng/ml, CA-19-9 13.5 U/ml
No family history of HNPCC

b) Sequencing the Tumor Cells and Using the ARID1A Gene as an at Least One Biomarker.

The sequencing of the BE3 and a comparative sample C3 were accomplished according to the before mentioned methods of nucleic acid preparation and PCR by using the primers P5 and P6 to identify if the biological sample BE3 and C3 have a mutation or not of the marker gene ARID1A.

TABLE 4
Identification of a mutation of the ARID1A
gene of biological example BE3

| | | | Sequence |
|---|---|---|---|
| | | | Identifi- |
| Sample | | | caton |
| Name | Name | Sequence 5′-3′ | Number |
| C3 | ARID1A | | SEQ ID |
| | wt | | NO: 9 |
| BE3 | ARID1A | | SEQ ID |
| | Q521* | | NO: 10 |
| P5 | ARID1A | GGTATGGTCAACAGGGCCAG | SEQ ID |
| | fw (F) | | NO: 11 |
| P6 | ARID1A | TCATACTTAGGAGGCCCAGG | SEQ ID |
| | rev (R) | | NO: 12 |

C3 is a comparative sample, the ARID1A wild type gene, which contains fully function and the sequence protocol SEQ-ID NO:9 showed no mutation of the marker gene ARID1A at nucleic acid position 826. The sequence protocol SEQ-ID NO:10 of BE3 showed a loss-of-function ARID1A^{Q521}* mutation at nucleic acid position 826.

c) Giving an Advice Whether to Start a Treatment

The sample BE3 showed an ARID1A^{Q521}* mutation, which is a loss-of-function mutation of the ARID1A gene. Therefore, a MEK inhibitor therapy, especially with trametinib, may be successful. The MEK inhibitor therapy should be initiated and the progress of the MEK inhibitor therapy should be monitored in regular time steps.

### EXAMPLE 4

Nucleic Acid Preparation

Genomic DNA from a biological sample BE4 and a comparative sample C4 were prepared using the QIAamp DNA Mini Kit (QIAGEN GmbH, QIAGEN Strasse 1, 40724 Hilden, Germany)) according to the manufacturer's protocols. The total DNA was eluted in 30 μl DNase/RNase-free distilled water (Thermo Fisher Scientific, Germany). Isolated DNA was quantified using a NanoDrop® ND-1000 Spectrophotometer (NanoDrop Technologies, 3411 Silverside Rd, 19819 Wilmington, United States of America) and stored at −20° C. until further use.

The combinations of primers that can be used for diagnosis are the forward and reverse primers of gene BMPR2. Though it will be expected that since the gene BMPR2, shown in SEQ-ID NO: 13, lie in a contiguous sequence the forward primer BMPR2 fw, shown in SEQ-ID NO: 15, and the reverse primer BMPR2 rev, shown in SEQ-ID NO: 16, can amplify a product.

PCR

The PCR reaction mixture (25 μl) contained 100 ng isolated DNA, 1× Phusion GC buffer (ThermoFisher Scientific, Germany), 3% DMSO, 200 μM dNTPs, about 0.5 μM of each primer, and about 0.02 U/μl of Phusion DNA polymerase (ThermoFisher Scientific, Germany). Amplification starts with initial denaturation at 95° C. for 5 min. Amplification protocol contains of denaturation at 95° C. for 30 sec, primer annealing at 58° C. for 30 sec and extension at 72° C. for 30 sec. Steps within amplification protocol are repeated 34 times in an automated DNA thermal cycler (Biometra TRIO Thermal Cycler Series, Analytik Jena AG, Germany). Final extension is performed at 72° C. for 5 min. The PCR products are resolved on 2% agarose gel stained with Ethidium bromide. PCR products are visualized under the UV trans illuminator and are expected to be 688 bp. Molecular size markers (GeneRuler™ 100 bp DNA Ladder, ThermoFisher Scientific, Germany) are run concurrently. PCR products were sequenced using BMPR2 fw SEQ-ID NO:15 and BMPR2 rev SEQ-ID NO:16 (LGCgenomics, Berlin, Germany). Sequencing results were analyzed using CLC genomic workbench (Qiagen Bioinformatics GmbH, QIAGEN Strasse 1, 40724 Hilden, Germany)).

According to the inventive method following steps were accomplished to determine whether to start a treatment of colorectal cancer comprising the provision of at least one inhibitor, preferably MEK inhibitor:

a) Information about the Biological Sample BE4 and the Comparative Sample C4

The biological sample BE4 and the comparative sample C4 were derived from the colon cancer of a Caucasian woman. At the time of surgery, the patient was 47 years old. The chemo-naive tumor was graded and staged as pT3, pN0 (0/22), M1(HEP), L0, V0, R0.
Histopathology: G2 UICC IV
Molecular Pathology: KRAS mutated
Localization: colon sigmoideum (60 cm ab ano)
Tumor marker: EA 1.0 ng/ml, CA-19-9 13.5 U/ml
No family history of HNPCC

b) Sequencing the Tumor Cells and Using the BMPR2 Gene as an at Least One Biomarker.

The sequencing of the BE4 and a comparative sample C4 were accomplished according to the before mentioned methods of nucleic acid preparation and PCR by using the primers P7 and P8 to identify if the biological sample BE4 and C4 have a mutation or not of the marker gene BMPR2.

TABLE 5
Identification of a mutation of the BMPR2
gene of biological example BE4

| | | | Sequence |
|---|---|---|---|
| | | | Identifi- |
| Sample | | | caton |
| Name | Name | Sequence 5′-3′ | Number |
| C4 | BMPR2 | | SEQ ID |
| | wt | | NO: 13 |
| BE4 | BMPR2 | | SEQ ID |
| | R873* | | NO: 14 |
| P7 | BMPR2 | TGCAGCAGAACCTCATGTGG | SEQ ID |
| | fw (F) | | NO: 15 |
| P8 | BMPR2 | ACACTGTGGTTTCTACCTGC | SEQ ID |
| | rev (R) | | NO: 16 |

C4 is a comparative sample, the BMPR2 wild type gene, which contains fully function and the sequence protocol SEQ-ID NO:13 showed no mutation of the marker gene ARID1A at nucleic acid position 826. The sequence protocol SEQ-ID NO:14 of BE4 showed a loss-of-function BMPR2^{R873}* mutation at nucleic acid position 826.

c) Giving an Advice Whether to Start a Treatment

The sample BE4 showed a BMPR2^{R873}* mutation, which is a loss-of-function mutation of the BMPR2 gene. Therefore, a MEK inhibitor therapy, especially with trametinib, may be successful. The MEK inhibitor therapy should be initiated and the progress of the MEK inhibitor therapy should be monitored in regular time steps.

1. A method for determining whether to start or to continue a treatment of cancer comprising the provision of at least one inhibitor, the method comprising:
a) measuring at least one characteristic of at least one biomarker in at least one biological sample comprising tumor cells of at least one tumor site of a tumor;
b) determining a loss or mutation of at least one marker gene in the at least one biological sample by use of the at least one biomarker;
c) the determining whether to start or to continue treatment with the at least one inhibitor if the measuring indicates that the tumor cells in the at least one biological sample comprise the at least one marker gene whose mutational status indicates a favorable outcome, whereby the at least one inhibitor is selected specifically in view of the determination of the mutational status of the at least one marker gene:
wherein the at least one marker gene is a tumor suppressor related to the activity of transforming growth factor-b/bone morphogenetic protein (TGF-b/BMR) pathway.

2. The method of claim 1, wherein the at least one characteristic is selected from the group consisting of size, sequence, composition, amount of the at least one biomarker, and a combination thereof.

3. The method of claim 1, wherein the at least one biomarker is a nucleic acid.

4. The method of claim 3, wherein the nucleic acid is selected from the group consisting of DNA, mRNA, cDNA, and any portion of any of the foregoing, wherein the portion corresponds to at least one mutation site of the at least one marker gene.

5. The method of claim 1, wherein the at least one inhibitor is a MEK inhibitor.

6. The method of claim 5, wherein the MEK inhibitor is selected from the group consisting of N-(3-{3-Cyclopropyl-5-[(2-fluor-4-iodphenyl)amino]-6,8-dimethyl-2,4,7-trioxo-3,4,6,7-tetrahydropyrido[4,3-d]pyrimidin-1(2H)-yl}phenyl)acetamid, [3,4-difluoro-2-[(2-fluoro-4-iodophenyl)amino]phenyl][3-hydroxy-3-(2S)-2-piperidinyl-1-azetidinyl]-methanone, 5-[(4-Bromo-2-chlorophenyl)amino]-4-fluoro-N-(2-hydroxyethoxy)-1-methyl-1H-benzimidiazol-6-carboxamide, and mixtures thereof.

7. The method of claim 1, wherein the at least one biological sample comprises tumor cells of at least two tumor sites of a tumor.

8. The method of claim 1, wherein the mutational status of the at least one marker gene is a mutation selected form the group consisting of deletion mutation, insertion mutation, frameshift mutant, nonsense mutant, missense mutant and splice mutant.

9. The method of claim 1, wherein the at least one marker gene is selected from the group consisting of mothers against decapentaplegic homolog 4 (SMAD4) gene, AT-rich interactive domain-containing protein 1A (ARID1A) gene, F-box/WD repeat-containing protein 7 (FBXW7) gene, and/or bone morphogenic protein receptor 2 (BMPR2) gene, Mitogen-activated protein kinase kinase (MEK) gene, and combinations thereof.

10. A biomarker to measure at least one characteristic of at least one marker gene in a biological sample, wherein the at least one biomarker corresponds to at least one marker gene which is a tumor suppressor related to the activity of transforming growth factor-β/bone morphogenetic protein (TGF-β/BMR) pathway.

11. The biomarker of claim 10, wherein the biological sample comprises tumor cells of at least one tumor site.

12. The biomarker of claim 10, wherein the at least one characteristic is selected from the group consisting of size, sequence, composition, amount, and a combination thereof.

13. The biomarker of claim 10, wherein the at least one biomarker is a nucleic acid selected from the group consisting of DNA, mRNA, cDNA, and any portion of any any portion of any of the foregoing, wherein the portion corresponds to at least one mutation site of the at least one marker gene.

14. The biomarker of claim 10, wherein the at least one marker gene is selected from the group consisting of mothers against decapentaplegic homolog 4 (SMAD4) gene, AT-rich interactive domain-containing protein 1A (ARID1A) gene, F-box/WD repeat-containing protein 7 (FBXW7) gene, and/or bone morphogenic protein receptor 2 (BMPR2) gene, Mitogen-activated protein kinase kinase (MEK) gene, and combinations thereof.

15. Use of at least one biomarker according to claim 10 in a method for determining whether to start or to continue a treatment of cancer with an at least one inhibitor.

16. The use of the at least one marker gene and the mutational status of the at least one marker gene as an at least one biomarker according to claim 10.

17. A kit for testing tumor response to inhibitor therapy comprising at least one reagent for detecting a loss-of-function mutation in nucleic acids isolated from at least one biological sample using at least one biomarker according to claim 10 which correlates to the at least one maker gene encoding a protein selected from the group consisting of mothers against decapentaplegic homolog 4 (SMAD4) gene, AT-rich interactive domain-containing protein 1A (ARID1A) gene, F-box/WD repeat-containing protein 7 (FBXW7) gene, and/or bone morphogenic protein receptor 2 (BMPR2) gene, Mitogen-activated protein kinase kinase (MEK) gene, and combinations thereof.

18. The kit of claim 17, wherein the at least one reagent is selected from the group consisting of nucleic acid sequences and primers.

19. A method of testing for a kinase inhibitor responsive solid tumor comprising detecting a loss of function in at least one marker gene encoding mothers against decapentaplegic homolog 4 (SMAD4), AT-rich interactive domain-containing protein 1A (ARID1A), F-box/WD repeat-containing protein 7 (FBXW7), bone morphogenic protein receptor 2 (BMPR2), Mitogen-activated protein kinase kinase (MEK), and combinations thereof, by contacting at least one biomarker isolated from at least one biological sample with a kit for testing for tumor response to inhibitor therapy according to claim 17.


SEQUENCE LISTING (4 sequences)

SEQ ID NO: 1 - DNA, 1659 bp, Homo sapiens
  Wildtyp
atggacaatatgtctattacgaatacaccaacaagtaatgatgcctgtctgagcattgtgcatagtttgatgtgccatagacaaggtggagagagtgaaacatttgcaaaaagagcaattgaaagtttggtaaagaagctgaaggagaaaaaagatgaattggattctttaataacagctataactacaaatggagctcatcctagtaaatgtgttaccatacagagaacattggatgggaggcttcaggtggctggtcggaaaggatttcctcatgtgatctatgcccgtctctggaggtggcctgatcttcacaaaaatgaactaaaacatgttaaatattgtcagtatgcgtttgacttaaaatgtgatagtgtctgtgtgaatccatatcactacgaacgagttgtatcacctggaattgatctctcaggattaacactgcagagtaatgctccatcaagtatgatggtgaaggatgaatatgtgcatgactttgagggacagccatcgttgtccactgaaggacattcaattcaaaccatccagcatccaccaagtaatcgtgcatcgacagagacatacagcaccccagctctgttagccccatctgagtctaatgctaccagcactgccaactttcccaacattcctgtggcttccacaagtcagcctgccagtatactggggggcagccatagtgaaggactgttgcagatagcatcagggcctcagccaggacagcagcagaatggatttactggtcagccagctacttaccatcataacagcactaccacctggactggaagtaggactgcaccatacacacctaatttgcctcaccaccaaaacggccatcttcagcaccacccgcctatgccgccccatcccggacattactggcctgttcacaatgagcttgcattccagcctcccatttccaatcatcctgctcctgagtattggtgttccattgcttactttgaaatggatgttcaggtaggagagacatttaaggttccttcaagctgccctattgttactgttgatggatacgtggacccttctggaggagatcgcttttgtttgggtcaactctccaatgtccacaggacagaagccattgagagagcaaggttgcacataggcaaaggtgtgcagttggaatgtaaaggtgaaggtgatgtttgggtcaggtgccttagtgaccacgcggtctttgtacagagttactacttagacagagaagctgggcgtgcacctggagatgctgttcataagatctacccaagtgcatatataaaggtctttgatttgcgtcagtgtcatcgacagatgcagcagcaggcggctactgcacaagctgcagcagctgcccaggcagcagccgtggcaggaaacatccctggcccaggatcagtaggtggaatagctccagctatcagtctgtcagctgctgctggaattggtgttgatgaccttcgtcgcttatgcatactcaggatgagttttgtgaaaggctggggaccggattacccaagacagagcatcaaagaaacaccttgctggattgaaattcacttacaccgggccctccagctcctagacgaagtacttcataccatgccgattgcagacccacaacctttagactga

SEQ ID NO: 2 - DNA, 1659 bp, Homo sapiens
  Loss-of-function mutation at postition 1082
atggacaatatgtctattacgaatacaccaacaagtaatgatgcctgtctgagcattgtgcatagtttgatgtgccatagacaaggtggagagagtgaaacatttgcaaaaagagcaattgaaagtttggtaaagaagctgaaggagaaaaaagatgaattggattctttaataacagctataactacaaatggagctcatcctagtaaatgtgttaccatacagagaacattggatgggaggcttcaggtggctggtcggaaaggatttcctcatgtgatctatgcccgtctctggaggtggcctgatcttcacaaaaatgaactaaaacatgttaaatattgtcagtatgcgtttgacttaaaatgtgatagtgtctgtgtgaatccatatcactacgaacgagttgtatcacctggaattgatctctcaggattaacactgcagagtaatgctccatcaagtatgatggtgaaggatgaatatgtgcatgactttgagggacagccatcgttgtccactgaaggacattcaattcaaaccatccagcatccaccaagtaatcgtgcatcgacagagacatacagcaccccagctctgttagccccatctgagtctaatgctaccagcactgccaactttcccaacattcctgtggcttccacaagtcagcctgccagtatactggggggcagccatagtgaaggactgttgcagatagcatcagggcctcagccaggacagcagcagaatggatttactggtcagccagctacttaccatcataacagcactaccacctggactggaagtaggactgcaccatacacacctaatttgcctcaccaccaaaacggccatcttcagcaccacccgcctatgccgccccatcccggacattactggcctgttcacaatgagcttgcattccagcctcccatttccaatcatcctgctcctgagtattggtgttccattgcttactttgaaatggatgttcaggtaggagagacatttaaggttccttcaagctgccctattgttactgttgatggatacgtggacccttctggaggagatcacttttgtttgggtcaactctccaatgtccacaggacagaagccattgagagagcaaggttgcacataggcaaaggtgtgcagttggaatgtaaaggtgaaggtgatgtttgggtcaggtgccttagtgaccacgcggtctttgtacagagttactacttagacagagaagctgggcgtgcacctggagatgctgttcataagatctacccaagtgcatatataaaggtctttgatttgcgtcagtgtcatcgacagatgcagcagcaggcggctactgcacaagctgcagcagctgcccaggcagcagccgtggcaggaaacatccctggcccaggatcagtaggtggaatagctccagctatcagtctgtcagctgctgctggaattggtgttgatgaccttcgtcgcttatgcatactcaggatgagttttgtgaaaggctggggaccggattacccaagacagagcatcaaagaaacaccttgctggattgaaattcacttacaccgggccctccagctcctagacgaagtacttcataccatgccgattgcagacccacaacctttagactga

SEQ ID NO: 3 - DNA, 22 bp, Artificial Sequence
  Primer 1 (SMAD4 fw 2)
aaattctcagttgacctggtcc

SEQ ID NO: 4 - DNA, 22 bp, Artificial Sequence
  Primer 2 (SMAD4 rev2 )
accgacaattaagatggagtgc